(19) United States
(12) Patent Application Publication
(10) Pub. No.: US 2026/0148830 A1
(43) Pub. Date: May 28, 2026

(54) Estimating Nutritional And Caloric Count of Food Using AI-Assisted Analysis of Food Photos

(71) Applicant: Katya Bakat Chapus, Los Angeles, CA (US)

(72) Inventor: Katya Bakat Chapus, Los Angeles, CA (US)

(21) Appl. No.: 18/893,920

(22) Filed: Sep. 23, 2024

Related U.S. Application Data
(60) Provisional application No. 63/584,546, filed on Sep. 22, 2023.
(60) Provisional application No. 63/657,711, filed on Jun. 7, 2024.

(51) Int. Cl. G16H 20/60; G06T 7/50; G06V 10/70; G06V 20/68; G06V 30/14; H04W 4/02; H04W 4/029

(52) U.S. Cl. CPC G16H 20/60; G06T 7/50; G06V 10/70; G06V 20/68; G06V 30/14; H04W 4/026; H04W 4/029; G06T 2207/10028; G06T 2207/20081; G06T 2207/30128

(57) ABSTRACT

Embodiments of the present disclosure may include a method of estimating the nutritional and caloric count of food based on a photo of the food, the method including taking a photo of the food. Embodiments may also include providing the photo of the food to an analyzer that includes Artificial Intelligence (AI) assistance, the AI having been trained on a variety of foods. Embodiments may also include analyzing the food by searching through a database of similar foods with AI assistance. Embodiments may also include estimating the caloric and nutritional value of the food. Embodiments may also include providing the estimated caloric and nutritional value of the food to a user.

### RELATED PATENT APPLICATIONS

This application claims the benefit of U.S. Provisional Patent Application Nos. 63/657,711, entitled “ESTIMATING NUTRITIONAL AND CALORIC COUNT OF FOOD USING AI-ASSISTED ANALYSIS OF FOOD PHOTOS” and 63/584,546, also entitled “ESTIMATING NUTRITIONAL AND CALORIC COUNT OF FOOD USING AI-ASSISTED ANALYSIS OF FOOD PHOTOS,” both of which are incorporated by reference herein.

### BACKGROUND

Various approaches have been developed for estimating the nutritional and caloric count of food based on different techniques. One common approach involves manual entry of food items into a database or application, where users input the food name, quantity, and other relevant information to obtain an estimate of the nutritional content. However, this method is time-consuming and prone to errors, as it relies on the user's ability to accurately identify and quantify the food consumed.

Another approach utilizes image recognition technology to estimate the nutritional content of food based on photos taken by users. These systems typically employ computer vision algorithms to identify the food items in the image and match them to a pre-existing database of food items with known nutritional values. While this approach eliminates the need for manual data entry, it often lacks accuracy and reliability due to variations in lighting conditions, angles, and presentation of the food.

Additionally, some existing methods employ machine learning techniques to estimate the nutritional content of food. These methods involve training a machine learning model on a variety of food images and their corresponding nutritional information. The model then uses this training data to predict the nutritional content of new food images.

However, these approaches often require extensive training datasets and may not accurately estimate the nutritional content of less common or unique food items. These approaches don't provide a comprehensive solution that combines the features described in this disclosure.

The present invention aims to overcome the limitations of existing methods by utilizing a combination of photo analysis, artificial intelligence assistance, and a database of similar foods to accurately estimate the nutritional and caloric count of food based on a photo taken by the user.

### SUMMARY OF THE INVENTION

In some aspects, the techniques described herein relate to a method of estimating the nutritional and caloric count of food based on a photo taken of the food, the method including the steps of: Taking a photo of the food; Providing the photo of the food to an analyzer that includes Artificial Intelligence assistance;

Analyzing the food by searching through a database of similar foods; Estimating the caloric and nutritional value of the food; Providing the estimated caloric and nutritional value of the food to a user.

In other aspects, embodiments of the invention may relate to one or more of the following:
Food Recognition
Volume and Nutrient Estimation
Tracking Consumption
GPS and Restaurant Database
Nutrition Label Scanning
Leftover Calculation
Calories Burned Integration
Ketosis Estimation
Goal Setting

Aspects of each may include:
Food Recognition: This requires advanced image recognition algorithms and possibly machine learning techniques to identify food from images. Volume and Nutrient Estimation: Once the food has been identified, estimating the volume will require depth image processing. The nutrient content can then be estimated using available food databases.
Tracking Consumption: The app will need to be able to store and retrieve user-specific information about food intake and nutritional content.
GPS and Restaurant Database: Using GPS to identify the restaurant will be straightforward, but building a database of every restaurant's offerings and their nutritional content could be challenging. Collaboration or partnerships with restaurants could make this process easier.
Menu Scanning: This requires Optical Character Recognition (OCR) technology to interpret the text in images, and then the same image recognition technology used for food can be applied.
Nutrition Label Scanning: Nutrition label scanning together with depth frame volume estimation will give very accurate nutrition and calorie estimates and the label scan associated with a photograph can be used to further improve our Als.
Leftover Calculation: This would require a way to compare before and after meal depth frame images and to estimate the change in volume. This could be quite challenging, depending on the type and arrangement of food.
Calories Burned Integration: If data from health or fitness apps that track calories burned is available, this could be integrated into the app.
Ketosis Estimation: Estimating ketosis from diet alone is not completely accurate, as it is influenced by many factors. However, a rough estimate could potentially be made based on the user's carbohydrate, protein, and fat intake.
Goal Setting: A more straightforward feature. The user would be able to input their personal goals which the app could use to provide feedback and suggestions.

Further aspects of the invention may include:
Algorithms for food identification using a smartphone camera,
Comprehensive databases for nutritional information,
Databases for restaurants,
Techniques for estimating food volume using depth frame images obtained from a smartphone camera, and
OCR (Optical Character Recognition) technologies suitable for smartphones, primarily for reading nutrition labels and menus.

Focusing now on particular aspects and possible combinations of elements, embodiments of the present disclosure may include a method of estimating the nutritional and caloric count of food based on a photo of the food, the method including taking a photo of the food. Embodiments may also include providing the photo of the food to an analyzer that includes Artificial Intelligence (AI) assistance, the AI having been trained on a variety of foods. Embodiments may also include analyzing the food by searching through a database of similar foods with AI assistance. Embodiments may also include estimating the caloric and nutritional value of the food. Embodiments may also include providing the estimated caloric and nutritional value of the food to a user.

Embodiments may also include analyzing the food includes recognizing the food using advanced image recognition algorithms and machine learning techniques. In some embodiments, the method according to may include estimating the volume of the food using depth image processing. Embodiments may also include depth image processing utilizes depth frame images obtained from a smartphone camera.

In some embodiments, the depth frame images may be further refined using data from a gravity sensor and a gyroscope to provide more accurate estimates of food volume and mass. In some embodiments, the method according to any may include tracking the consumption of food by storing and retrieving user-specific information about food intake and nutritional content.

In some embodiments, the method according to any may include identifying the establishment serving the food using GPS data. In some embodiments, the establishment may be a restaurant, and the method may include referencing a database of restaurant nutritional data. In some embodiments, the method according to any may include scanning a menu for information about the food to be consumed using Optical Character Recognition (OCR) technology.

Embodiments may also include scanning a menu includes interpreting text in images and using image recognition to identify food items. In some embodiments, the method according to any may include scanning a nutrition label and employing depth frame volume estimation to improve caloric and nutrition information. In some embodiments, the method according to any may include analyzing leftovers by comparing an image of the food prior to consumption and an image of the food remaining after consumption to estimate the change in volume.

Embodiments may also include analyzing leftovers includes using depth image processing to accurately estimate the volume of food consumed. In some embodiments, the method according to any may include tracking a user's consumption of calories with a fitness app. In some embodiments, the method according to any may include estimating ketosis status based on the user's carbohydrate, protein, and fat intake. In some embodiments, the method according to any may include inputting personal user goals and providing feedback based on the user's nutritional intake.

Embodiments of the present disclosure may also include a system for estimating the nutritional and caloric count of food, including a camera for taking photos of food. Embodiments may also include an analyzer including AI assistance for analyzing the food. Embodiments may also include a database of similar foods with known nutritional values. Embodiments may also include a module for estimating the caloric and nutritional value of the food. Embodiments may also include a module for providing the estimated caloric and nutritional value to a user.

In some embodiments, the analyzer includes advanced image recognition algorithms and machine learning techniques for identifying food from images. In some embodiments, the camera may be a smartphone camera capable of capturing depth frame images for use in depth image processing. In some embodiments, the system according to may include a gravity sensor and a gyroscope to refine the depth frame images and provide more accurate estimates of food volume and mass.

The foregoing may be used separately or in combination. That is, the invention is not limited to the steps as explicitly recited, and combinations of the foregoing are within the scope of the invention.

### BRIEF DESCRIPTION OF THE FIGURES

FIG. 1 illustrates a person taking a photo of food according to one embodiment of the present application;

FIG. 2 is a flowchart illustrating a method of estimating the nutritional and caloric count of food, according to some embodiments of the present disclosure.

FIG. 3 is a flowchart further illustrating the method of estimating the nutritional and caloric count of food from FIG. 1 according to some embodiments of the present disclosure.

FIG. 4 is a block diagram illustrating a system, according to some embodiments of the present disclosure.

FIG. 5 is a block diagram further illustrating an aspect of an embodiment of the system.

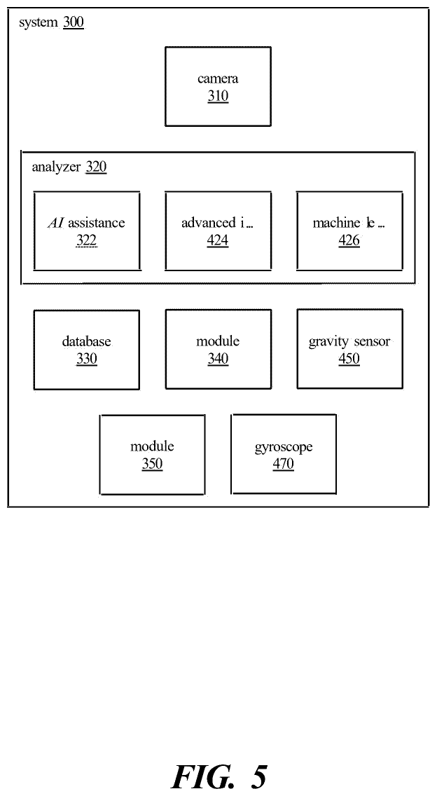

FIGS. 6A-6D illustrate example screens that may be displayed to a user in an embodiment of the present system, including a login screen, a main screen, a food capture screen, and a nutrition label screen.

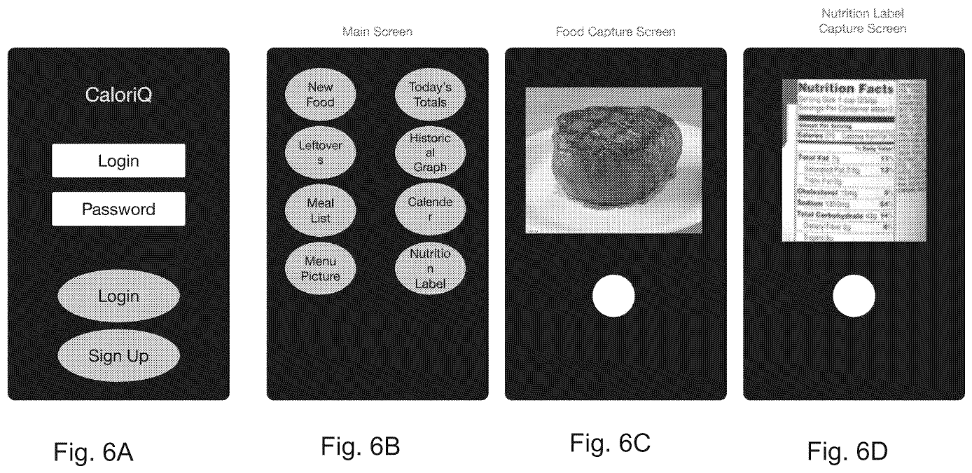

FIGS. 7A-D illustrate example screens that may be displayed to a user in an embodiment of the present system, including a food list screen, food nutrition breakdown screen, a leftover capture screen, and a meal list screen.

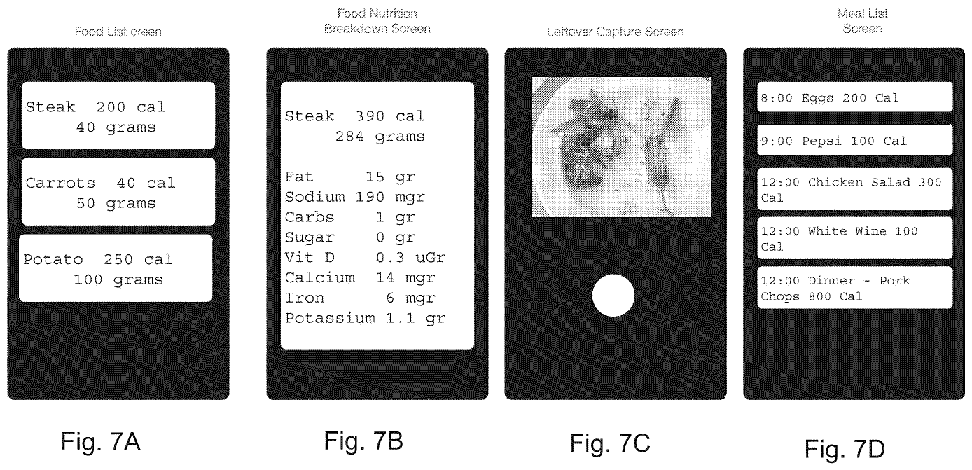

FIGS. 8A-8C illustrate example screens that may be displayed to a user in an embodiment of the present system, including a menu usage screen, a nutrition breakdown screen, and a menu capture screen.

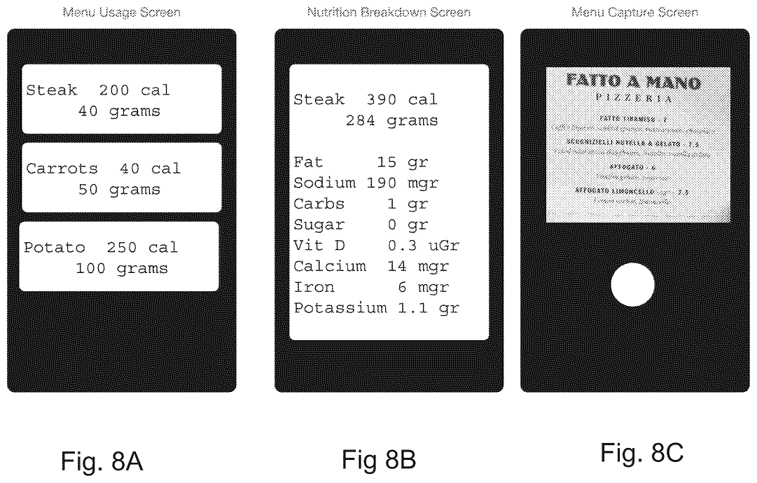

FIGS. 9A-9D illustrate example screens that may be displayed to a user in an embodiment of the present system, including a nutrition label usage screen, a daily/weekly/monthly calorie-nutrition tracking screen, a historical graphs nutrient screen, and a calorie/nutrition calendar historical tracking screen.

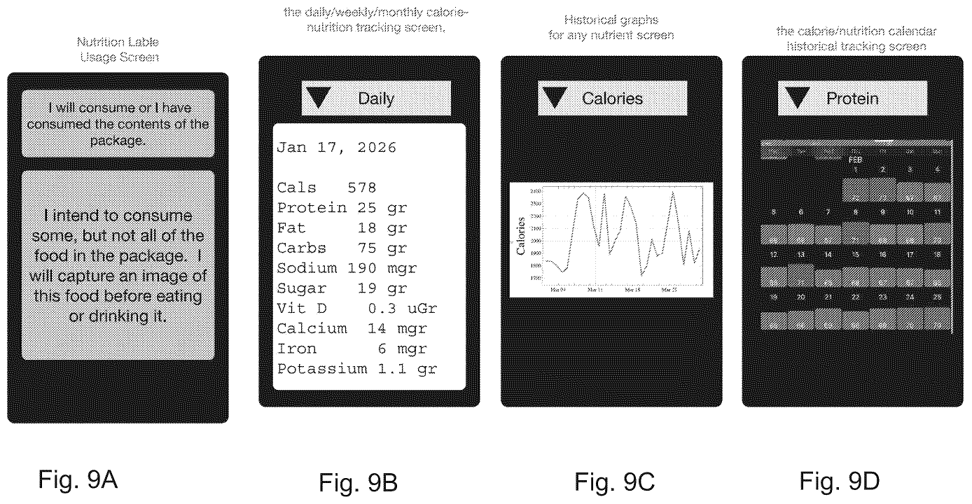

### DETAILED DESCRIPTION

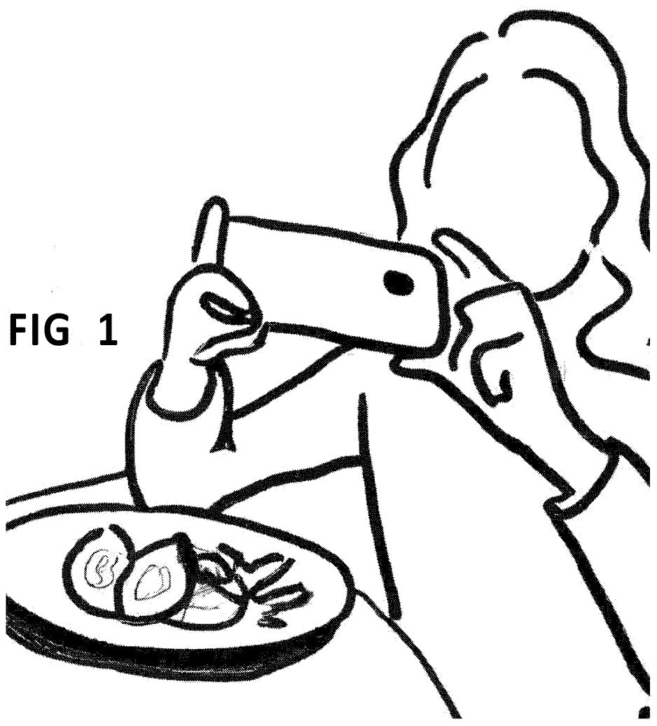

FIG. 1 is a flowchart that describes a method of estimating the nutritional and caloric count of food, according to some embodiments of the present disclosure. In some embodiments, at 110, the method may include taking a photo of the food. At 120, the method may include providing the photo of the food to an analyzer that includes Artificial Intelligence (AI) assistance, the AI having been trained on a variety of foods. At 130, the method may include analyzing the food by searching through a database of similar foods with AI assistance. At 140, the method may include estimating the caloric and nutritional value of the food. At 150, the method may include providing the estimated caloric and nutritional value of the food to a user.

In some embodiments, analyzing the food may include recognizing the food using advanced image recognition algorithms and machine learning techniques. In some embodiments, the method may include estimating the volume of the food using depth image processing. In some embodiments, depth image processing may utilize depth frame images obtained from a smartphone camera. In some embodiments, the depth frame images may be further refined using data from a gravity sensor and a gyroscope to provide more accurate estimates of food volume and mass.

In some embodiments, scanning a menu may include interpreting text in images and using image recognition to identify food items. In some embodiments, analyzing leftovers may include using depth image processing to accurately estimate the volume of food consumed.

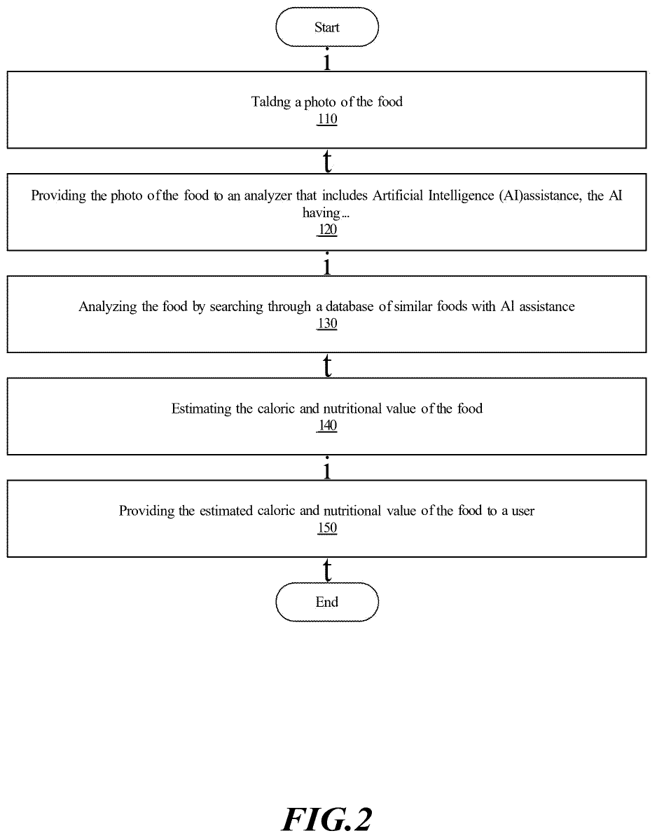

FIG. 2 is a flowchart that further describes the method of estimating the nutritional and caloric count of food from FIG. 1, according to some embodiments of the present disclosure. In some embodiments, the establishment may be a restaurant. At 220, the method may include referencing a database of restaurant nutritional data.

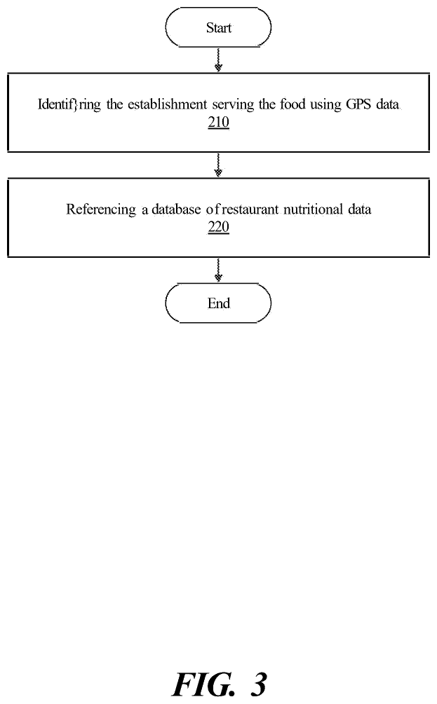

FIG. 3 is a block diagram that describes a system 300, according to some embodiments of the present disclosure. In some embodiments, the system 300 may include a camera 310 for taking photos of food, an analyzer 320, a database 330 of similar foods with known nutritional values, a module 340 for estimating the caloric and nutritional value of the food, and a module 350 for providing the estimated caloric and nutritional value to a user. The analyzer 320 may include AI assistance 322 for analyzing the food.

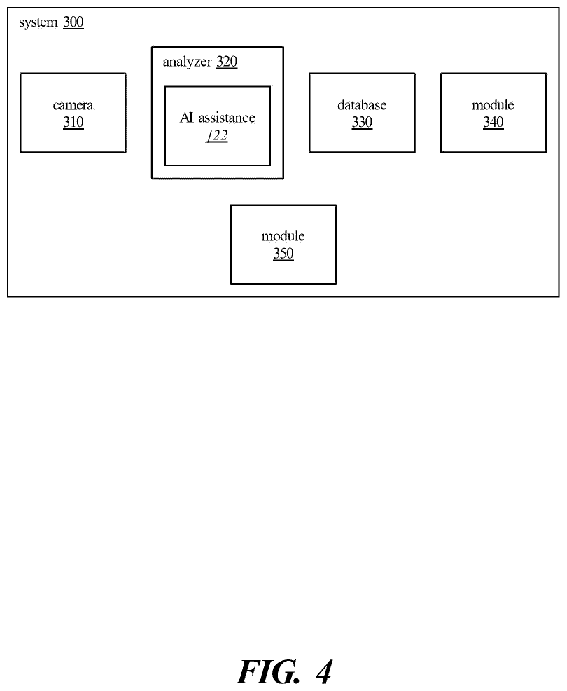

FIG. 4 is a block diagram that further describes the system 300 from FIG. 3, according to some embodiments of the present disclosure. In some embodiments, the analyzer 320 may include advanced image recognition algorithms 424 and machine learning techniques 426 for identifying food from images. In some embodiments, the camera 310 may be a smartphone camera capable of capturing depth frame images for use in depth image processing. In some embodiments, the system 300 may include a gravity sensor 450 and a gyroscope 470 to refine the depth frame images and provide more accurate estimates of food volume and mass.

### Other Aspects

In one embodiment, depth information, the gravity sensor, and the gyroscope in the iPhone and other sophisticated mobile phones may be used. The depth frame in many iPhones and some other mobile phones provides information about the distance between the camera and the objects in the picture.

The gyroscope and gravity sensor provide information about the orientation of the camera. This provides more accurate estimates of the food volume, mass, and nutritional content. This approach is in contrast to approaches in which calorie apps use just the two-dimensional RGB (red-green-blue) camera information. These can be fooled.

In some instances, we believe other camera apps can be off by as much as 700%. For example, if you take a mixing bowl and fill it with 8 cups of whole milk, some apps give poor calorie estimates. In one test, a prototype of an embodiment of our software gave the correct calorie count of 1200 calories (plus or minus 50 calories). Other apps we tested gave an answer of less than 200 calories.

Similar errors may occur when a small plate is used, a large mug, or if no plate at all is present. In some apps, the same calorie and/or other data estimate is made for a small banana as for a large banana. These approaches may assume the user is eating an integer number of servings rather than estimating the volume and mass which is what our software does.

In addition, embodiments of our approach as coded into software have been able to estimate the amount of food consumed if the eater only eats part of the food on their plate and they then take a picture of the leftovers. By combining the depth sensor and AI, our software calculated the amount eaten.

We are not aware of prior art that uses the depth sensor or that can estimate the calorie difference between the original plate of food and the plate of leftovers after the meal.

We are also not aware of prior art approaches that read menus. An aspect of one embodiment of our approach is that our software reads menus including any calorie information on the menu and it can even use descriptions of the food in the menu to better identify foods on the plate.

One embodiment of our approach can use GPS information to locate the restaurant or other establishment in which the photo of the food is taken. We can create or update a database that includes the calorie and other nutrition information of foods at every restaurant where our future customers eat.

Further technical information about our approach is attached hereto. Non-limiting examples of potential screens in one embodiment of the present invention are also attached hereto.

It is understood that the foregoing is merely illustrative of the invention, and that the scope of the invention may be more fully understood with reference to the Claims and associated Drawings.

### 2 The Application

### 2.1 List of Screens

The application consists of the following screens:
1. The login screen.
2. the main screen,
3. * the food capture screen,
4. the food list screen,
5. the food nutrition breakdown screen.
6. * the leftover capture screen
7. * the meal list screen
8. * the menu capture screen
9. (Optional) the menu usage screen
10. * the nutrition label capture screen.
11. (Optional) the nutrition label information screen.
12. the nutrition label usage screen.
13. * the daily/weekly/monthly calorie/nutrition tracking screen,
14. * Historical graphs for any nutrient screen.
15. Nutrient choice screen.
16. * the calorie/nutrition calendar historical tracking screen, and
17. * the settings screen.

The screens marked with a * can be accessed from the main screen. The main screen has a button for each of these options.

2.1.1 Screen details

Food capture screen: The food capture screen always starts in acquisition mode. In acquisition mode, the image changes to show what the camera sees and there are two buttons: a capture button and a cancel button. The cancel button brings the user back to the main screen. The capture button captures an image of the food and puts the screen in accept/reject mode. In accept/reject mode, there are two buttons “accept” and “reject”. Reject puts the screen back on acquisition mode. Accept takes the user to the food nutrition breakdown screen if one food or drink is detected or to the food list screen if more than one food is detected.

Food list screen: If multiple foods are detected, then the food list screen lists all the foods detected along with their weights, volumes, and calories. The weight, volume, and calories can be edited. There are accept and or reject, buttons. Pushing either of there buttons brings the user back to the main screen. Also, if the user clicks on the food item, lie or she will be taken to the food nutrition breakdown screen for that food. If the user subsequently exits from the food nutrition breakdown screen, they will be returned to the food list screen.

Nutrition label usage screen: The nutrition label usage screen has the following options:
1. I will consume or I have consumed the contents of the package.
2. I intend to consume some, but not all of the food in the package. I will capture an image of this food before eating or drinking it.

Food nutrition breakdown screen: The food nutrition breakdown screen is displayed after single food item or drink is captured. It can also be accessed from the meal list screen if a single food has been imaged, or from the food list screen. This screen shows a single type of food at the top followed by the weight, volume, and calories for the amount of that food that was imaged. After those values, fats, proteins, fiber, vitamins, and minerals are displayed for the amount of this particular type of food given in one image or obtained by scanning a nutrition label.

Historical graphs for any nutrient screen By default this screen shows daily calorie consumption on the vertical axis and a month of days on the horizontal axis (day of the month). The user may choose any nutrient by pushing a “change nutrient” button which takes them to the Nutrient choice screen. The user can choose to view either the last week, the last month, or the last year. If the user chooses the last year, the average calories (or other nutrient) is shown per clay.

Nutrient choice screen: This screen just shows a scrollable list consisting of {calories, carbs, protein, fat}, a list of vitamins, and a list of minerals. The user can choose any one of these to return to the previous screen.

### 2.2 Main Usage Path

The main usage method for the application is listed below.
1. The user sits down and is ready to eat a meal.
2. The user logs into the application on their device (usually a mobile phone with a 3D imaging camera).
3. After the user logs in, she or he will be at the main screen.
4. The user chooses to go the food capture screen.
5. The user aims the camera at a plate of food or a drink and captures the image of the food or beverage.
6. After the user takes the image, she or he will be at the food nutrition breakdown screen which shows the weight or volume of each type of fox on the plate or the weight of the drink. In addition the user can scroll downward to see the nutrition information for each food item or for the drink. The user can alter these values if they wish to do so.
7. The user must choose to either accept or reject the results.
8. The user is then returned to the main screen.
9. At the end of the meal, if there are any leftover foods or leftover drink, then the user can choose to go to the leftover capture screen
10. The user would then point the camera at the leftovers and capture an image of the leftovers
11. After the user captures the image of the leftovers, she or he will be sent to the food nutrition breakdown screen, but the word “leftovers” will be visible. All nutrition values and weights will be in shown. The user can edit these values.
12. The user must choose to either accept or reject the results. If accepted, the nutrition values will be subtracted from the daily totals.
13. The user is then returned to the main screen.

### 2.3 Other Usage Paths

### 2.3.1 Nutrition Review

Any time that the user is at the main screen, they can choose to view their consumed nutrients by going to the daily/weekly/monthly calorie/nutrition tracking screen or the calorie/nutrition calendar historical tracking screen. Once they are done reviewing their nutrition information, they will push a but ton to return to the main screen.

2.3.2 Consuming Prepackaged Foods with a Label

If the user is eating an entire package of food like a frozen dinner, then they follow this process.
1. The user goes to the label capture screen.
2. The user points the camera at the label and captures the image of the label.
3. After the label image is captured the user will be at the nutrition label usage screen.
4. The user then selects “I will consume or I have consumed the contents of the package.”
5. The user is then returned to the main menu.

If the user is eating only a portion of a package of food like some soup, or heating up some pizza, then they follow this process.
1. The user goes to the label capture screen.
2. The user points the camera at the label and captures the image of the label.
3. After the label image is captured the user will be at the nutrition label usage screen.
4. The user then selects “I intend to consume some, but not all of the food in the package. I will capture an image of this food before eating or drinking it.”
5. The user is then sent to the food capture screen.
6. The user aims the camera at a plate of food or a drink and captures the image of the food or beverage.
7. After the user takes the image, she or he will he at the food nutrition breakdown screen which shows the weight or volume of each type of food on the plate or the weight of the drink. In addition the user can scroll downward to see the nutrition information for each food item or for the drink. The user can alter these values if they wish to do so.
8. The user must choose to either accept or reject the results.
9. The user is then returned to the main screen.
10. At the end of the meal, if there are any leftover foods or leftover drink, then the user can choose to go to the leftover capture screen
11. The user would then point the camera at the leftovers and capture an image of the leftovers
12. After the user captures the image of the leftovers, she or he will be sent to i he food nutrition breakdown screen, but the word “leftovers” will be visible. All nutrition values and weights will be in shown. The user can edit these values.
13. The user must choose to either accept or reject the results. If accepted, the nutrition values will be subtracted from the daily totals.
14. The user is then returned to the main screen.

### 2.3.3 Using the Menu at a Restaurant

In order to record nutrition information at a restaurant, the user follows this sequence
1. The user has chosen an item, a meal, or a drink on the menu
2. The user chooses the menu capture option on the main screen
3. The user points the camera at the item on the menu that they want to eat and takes a picture.
4. The user is returned to the main screen.
5. The user is then sent to the food capture screen.
6. The user aims the camera at a plate of food or a drink and captures the image of the food or beverage.
7. After the user takes the image, she or he will he at the food nutrition breakdown screen which shows the weight or volume of each type of food on the plate or the weight of the drink. In addition the user can scroll downward to see the nutrition information for each food item or for the drink. The user can alter these values if they wish to do so.
8. The user must choose to either accept or reject the results.
9. The user is then returned to the main screen.
10. At the end of the meal, if there are any leftover foods or leftover drink, then the user can choose to go to the leftover capture screen
11. The user would then point the camera at the leftovers and capture an image of the leftovers
12. After the user captures the image of the leftovers, she or he will be sent to i he food nutrition breakdown screen, but the word “leftovers” will be visible. All nutrition values and weights will be in shown. The user can edit these values.
13. The user must choose to either accept or reject the results. If accepted, the nutrition values will be subtracted from the daily totals.
14. The user is then returned to the main screen.

### 3 Algorithms

### 3.1 Introduction

We use several algorithms for food volume detection and food identification. They are described in this section.

### 3.2 SVD Table or Plate Estimation

Often we will want to find the equation of the “best-fit” plane through a set of points. The set of points will often be a table or the unobscured part of a plate. We use the following well-known in the art algorithm for fitting the plane.
Find several points (preferably more than 10) that are on the part, of the table or plate. The points have the form (xᵢ,yᵢ,zᵢ) for i=1, 2, . . . ,n.
(optional) The set of points

$$\{ ( {x}_{i} , {y}_{i} , {z}_{i} ) \vert i ∈ \{ 1 , 2 , … , n \} \}$$

may be centered and scaled by finding the Z-scores for each entry by subtracting the mean and dividing by the standard deviation. This is a well-known technique.
Form the matrix

$$A = ( \begin{matrix}1 & {x}_{1} & {y}_{1} & {z}_{1} \\ 1 & {x}_{2} & {y}_{2} & {z}_{2} \\ ⋮ & ⋮ & ⋮ & ⋮ \\ 1 & {x}_{n} & {y}_{n} & {z}_{n}\end{matrix} ) .$$

Find the Singular Value Decomposition of A

$${UDV}^{T} = A$$

where U and V are unitary and D is diagonal with decreasing singular values.
Lert: v₄ be the fourth column of V.
The quadratic surface is

$$( 1 , x , y , z ) \cdot {v}_{4} = 0.$$

If the Z scores were computed in the optional step above, then the inverse linear transformation is applied to map the surface calculated in the previous step back into the original coordinate system.

### 3.3 SVD Quadratic Surface Estimation

We use the following well-known in the art algorithm for fitting a quadratic surface. We apply this algorithm to find the “best-fit” quadratic surface of a food or part of a food.
Find several points (preferably more than 100) that are on the part of the food which is quadratically shaped (usually spherical or ellipsoidal, but portions of the food surface may be a paraboloid, hyperboloid, or a cone). The points have the form (xᵢ,yᵢ,zᵢ) for i=1, 2, . . . , n.
(optional) The set of points

$$\{ ( {x}_{i} , {y}_{i} , {z}_{i} ) \vert i ∈ \{ 1 , 2 , … , n \} \}$$

may be centered and scaled by finding the Z-scores for each entry by subtracting the mean and dividing by the standard deviation. This is a well-known technique.
Form the matrix

$$A = ( \begin{matrix}1 & {x}_{1} & {y}_{1} & {z}_{1} & {x}_{1} {y}_{1} & {x}_{1} {z}_{1} & {y}_{1} {z}_{1} & {x}_{1}^{2} & {y}_{1}^{2} & {z}_{1}^{2} \\ 1 & {x}_{2} & {y}_{2} & {z}_{2} & {x}_{2} {y}_{2} & {x}_{2} {z}_{2} & {y}_{2} {z}_{2} & {x}_{2}^{2} & {y}_{2}^{2} & {z}_{2}^{2} \\ ⋮ & ⋮ & ⋮ & ⋮ & ⋮ & ⋮ & ⋮ & ⋮ & ⋮ & ⋮ \\ 1 & {x}_{n} & {y}_{n} & {z}_{n} & {x}_{n} {y}_{n} & {x}_{n} {z}_{n} & {y}_{n} {z}_{n} & {x}_{n}^{2} & {y}_{n}^{2} & {z}_{n}^{2}\end{matrix} ) .$$

Find the Singular Value Decomposition of A

$${UDV}^{T} = A$$

where U and V are unitary and D is diagonal with decreasing singular values.
Let v₁₀ be the tenth column of V.
The quadratic surface is

$$( 1 , x , y , z , xy , xz , yz , {x}^{2} , {y}^{2} , {z}^{2} ) \cdot {v}_{10} = 0.$$

If the Z scores were computed in the optional step above, then the inverse linear transfor-nation is applied to map the surface calculated in the previous step back into the original coordinate system.

### 3.4 Gravity Alignment

Many of our algorithms benefit from changing the coordinate system so that gravity is downward. We have three methods for detecting a gravity vector:
1. detecting the table or plate and assuming that gravity is perpendicular to the plane of the table or the plane of the plate using the SVD technique from section 3.2,
2. get ting gravity data from the phone, and
3. a combination of the two techniques above using inverse error variance weighting. (See e.g. the Wikipedia.)

Once the gravity vector is normalized to v=(x, y, z) where ∥v∥₂=1, we find the rotation matrix

$$R = ( \begin{matrix}\frac{{y}^{2} ( 1 - {x}^{2} z ) - {x}^{2} z ( {x}^{2} + {z}^{2} )}{{x}^{2} + {y}^{2}} & - \frac{xy ( {x}^{2} z + {y}^{2} z + {z}^{3} + 1 )}{{x}^{2} + {y}^{2}} & x \\ - \frac{xy ( {x}^{2} z + {y}^{2} z + {z}^{3} + 1 )}{{x}^{2} + {y}^{2}} & \frac{{x}^{2} ( 1 - {y}^{2} z ) - {y}^{2} z ( {y}^{2} + {z}^{2} )}{{x}^{2} + {y}^{2}} & y \\ - x & - y & - z\end{matrix} )$$

and apply that isometry to every vertex in the depth frame.

### 3.5 Ellipsoidal Food Volume Detection

### 3.5.1 Main Ellipsoidal Algorithm

Ellipsoidal food volume detection is used for oranges, hard-boiled eggs, lemons, apples, whole grapefruits, individual grapes, and anything else that has an ellipsoidal or spherical shape. The process is:
1. Identify the table plane. (Or the plane going through the bottom of the plate. See section 3.2.)
2. Rotate all vertices in the triangular mesh of the food so that the z coordinate points upward perpendicular to the table. (See section 3.4.)
3. Translate the z coordinates of all points with the formula,

$${z}_{new} = {z}_{oid} - {z}_{table}$$

where the plane of the table (or plate) is z_{table}. The resulting matrix of n row vectors corresponding to each vertex is called V∈R^{n×3}. Using gradient descent, find the parameters h, r, x₀, and y₀ which minimize the sum of the squared distances from every food vertex to the ellipsoid

$$\frac{{z}^{2}}{{h}^{2}} + \frac{{( x - {x}_{0} )}^{2} + {( y - {y}_{0} )}^{2}}{{r}^{2}} = 1.$$

More specifically, minimize

$$Error = {\sum }_{i = 1}^{n} {d ( {v}_{i} , E )}^{2}$$

where E is the manifold defined by (1), and d is the Euclidean distance The starting point for the iterations should be h=maxᵢ(zᵢ)/2, r equals the largest eigenvalue of the matrix M^{T}M where m_{i,j}=v_{i,j}−μⱼ, i=1, . . . n, j∈1, 2,

$${μ}_{i} = \frac{1}{n} {\sum }_{i} {v}_{i , j} ,$$

the vectors vᵢ∈R³ are the rows of V, x₀=μ₁, and y₀=μ₂.
4. Once the ellipsoid is determined by minimizing the Error, the volume of the ellipsoid is merely

$$Vol = \frac{4 π {r}^{2} h}{3} .$$

### 3.5.2 Cut Ellipsoid

If an ellipsoid is cut we use the Quadratic Surface Fit Algorithm from section 3.3 to approx-imate the ellipsoid and the SVD plane estimation algorithm in section 3.2 to find the cutting plane if it is visible. At that point the volume of the cut ellipsoid is estimated by applying the appropriate linear transformation L needed to make the ellipsoid a sphere centered at (0,0,0). The volume of the cut sphere is

$$VolumeOfCutSphere = \frac{1}{3} {π ( h - r )}^{2} ( h + 2 r )$$

where h is the distance from the cutting plane¹ to (0,0,0). Now the volume of the cut ellipsoid is the VolumeOfCutSphere divided by the determinant of L. ¹The new cutting plane for the sphere is the L applied to the old cutting plane of the ellipsoid.

### 3.6 Volume From a Triangular Mesh

We represent a triangular mesh with two matrices V∈R^{m×3} and M∈{1, 2, . . . , m}^{n×3} where m is the number of vertices and n is the number of triangles. We assume that the triangles are oriented outward, meaning that the vector u_{1,i}×u_{2,i} points outward away from the food for i=1, 2 . . . , n where

$$\begin{matrix}{u}_{1 , i} = V ( {M}_{i , 2} ) - V ( {M}_{i , 1} ) , \\ {u}_{2 , i} = V ( {M}_{i , 3} ) - V ( {M}_{i , 2} ) , \\ {u}_{3 , i} = V ( {M}_{i , 1} ) - V ( {M}_{i , 2} ) .\end{matrix} and$$

$$Let {z}_{i} = \frac{1}{3} {\sum }_{j = 1}^{3} V ( M ( j , i ) , 3 ) .$$

### 3.6.1 Watertight Mesh

If the triangular mesh “watertight” then the enclosed volume is

$$volume = {\sum }_{i = 1}^{n} ( {u}_{1 , i} \times {u}_{2 , i} ) \cdot ( {z}_{i} , 0 , 0 ) .$$

### 3.6.2 Resting on a Table or Plate

Often we will only see the top of the food which is resting on a table. If the coordinate system is oriented so that the vector (0,0,−1) points downward, and the Table (or Plate) has equation z=h for a fixed real number h, then

$$volume = {\sum }_{i = 1}^{n} ( {u}_{1 , i} \times {u}_{2 , i} ) \cdot ( {z}_{i} - h , 0 , 0 )$$

### 3.7 Depth-Color Image Segmentation

We use a combination of standard techniques for image segmentation including Edge Detection (Sobel, Canny, or Prewitt), Watershed, Bilateral Filtering, Graph-Cut Algorithms, Markov Random Fields, and Convolutional Neural Nets; however, we will often add depth as a separate channel or use depth gradients to aid in Edge Detect ion.

### 3.7.1 Distance Transform

When using depth to aid image segmentation, we will often emphasize the depths corresponding to food with the transformation

$${depth}_{new} = 255 \cdot \frac{1}{π} ( arctan ( \frac{{depth}_{old} - {μ}_{food}}{{σ}_{food}} ) + \frac{π}{2} )$$

where μ_{food} is the average distance from the camera of the food vertices and σ_{food} is the sample standard deviation of those depths.

### 3.7.2 Using EM, Markov Random Fields, and Neural Nets for Image Segmentation

One method that we have used for image segmentation is a variation of the well known EM/Markov Random Fields method. When applying the Markov Random Fields method, each type of food is associated with a probability distribution of RGB colors. We then optimize the probability of the image by classifying each pixel by maximizing the probability that it is in the given color distribution given the classification of neighboring pixels. We added three innovations to this process.
1. using a convolutional neural net to classify the pixels.
2. using depth gradients to adjust the probability that neighboring pixels are the same food. (High depth gradient implies that the pixels are more likely to be different foods.)
3. some foods are less smooth than others, so, we can use that fact to adjust the probability that neighboring pixels are the same food rather than only relying on the depth gradient for this probability.

### 3.8 Applying Word to Vector and Picture to Vector

We often use AIs that will map both text and images to the same n-dimensional feature space. This is one of our main ways of classifying food. The method we use is to look at the cosine distance from the food image vector to the word vector for a long list of food and choose the foods with the highest probability.

### 3.8.1 Food Clustering

Also, we break down the food databases into groups by using k-means with Akaike information criterion to create food groups.

### 3.9 Top Down Food Detection

One of our main methods for food detection is to go from the top down. When using this technique, we follow these steps:
1. First we establish a gravity vector as in section 3.4 and apply the rotation matrix R to every vertex.
2. We then run image segmentation on the depth frame image (see section 3.7).
3. We find the segment with the highest z coordinate.
4. We identify the food in this highest segment. (See section 3.8.)
5. Next, we do the same food identification for every other segment that neighbors highest segment to see if we get the same food. We often penalize the neighboring segment using fuzzy logic if there is a sharp drop in distance on the boundary between the segments. We used this method to grow the segment into a connected group of segments representing the food.
6. We remove the segments corresponding to the highest food, and repeat the process with the second highest segment.
7. We repeat the process of the previous step until we reach the plate or table.

### 3.9.1 Foods at the Same Height are Often the Same

Another technique that we use is to look at separate connected components of segments that are the same height. Often there can be several pieces of the same food found at the same height. This aids in classification. For example, there may be several baby carrots on a plate which are separated by air, but all have the same height.

### 3.10 Iterative Closest Point for Shape Fitting and Platonic Ideal Foods

We have been experimenting with using the iterative closest point method to fit the shape of a food. For example, we create an “average” red delicious apple by resizing all of the red delicious apples in our database to the average size. Then we perform 3 dimensional image registration to align all the apples. Then we average all of these apple surfaces. This gives us an idealized Platonic apple.

Now when we find a red delicious apple in a depth frame, we can use the iterative closest point method to align the apple in the depth frame with our idealized Platonic apple. We do insert a least squares resizing step into the iterative closest point method to allow for smaller or larger apples. This method yields a much more accurate estimate of the apple volume without the need to take pictures from multiple angles.

3.10.1 Combining Iterative Closest Point with Parameter Estimation for Fitting a Shape Model of the Food.

A similar but more flexible method can be used by adding parameters to the Platonic ideal. For example, not all bananas have the same curvature and thickness, so we could add parameters for the curvature of the banana and the thickness of the banana. We would then insert, the step of finding the maximum likelihood estimator of the curvature and the thickness of the banana. Unfortunately, this method requires the construction of a function that maps the ordered triple (size, curvature, thickness) into the space of 2-dimensional manifolds representing a banana which requires a bit of effort for each of these parametrized models, so, at this point, this method is only experimental.

### 3.11 Applied Symmetry

When an object is viewed from one side, we can't see the other side. If the top of the object has symmetry (like a bowl, a sandwich, a cake, or a cookie), we can reconstruct the other side using symmetry. We look for rotational and reflective symmetry to do this operation.

### 3.12 Using Subsections for Multiple Food Identification

We also have tried breaking the image down into overlapping squares and running food identification on each square. This is a well-known technique.

1-20. (canceled)

21. A computer-implemented method of estimating a nutritional and caloric content of food, comprising:
acquiring, by a depth-frame camera of a mobile computing device, a color image and a depth frame image of a food item, the depth frame image comprising depth values representing distances between the depth-frame camera and respective surface points of the food item;
acquiring, by an inertial measurement unit of the mobile computing device, orientation data comprising gravity vector data from a gravity sensor and angular rate data from a gyroscope;
determining, by a processor of the mobile computing device, a gravity-aligned coordinate system by computing a rotation matrix from the gravity vector data and applying the rotation matrix to depth frame coordinates of the depth frame image;
identifying, by the processor, a planar reference surface in the gravity-aligned coordinate system by applying a plane-fitting algorithm to a plurality of depth frame points corresponding to at least one of a table surface or a plate surface supporting the food item;
estimating, by the processor, a three-dimensional volumetric measurement of the food item by computing, in the gravity-aligned coordinate system, a volume defined by food surface points above the identified planar reference surface;
identifying the food item by applying a trained machine learning classifier to at least the color image;
determining a caloric and nutritional estimate for the food item by querying a nutritional database using the identified food item and the estimated volumetric measurement; and
outputting the caloric and nutritional estimate to a user interface of the mobile computing device.

22. The method of claim 21, wherein identifying the planar reference surface comprises applying a Singular Value Decomposition (SVD) algorithm to a matrix formed from coordinates of the plurality of depth frame points to determine a best-fit plane equation for the table surface or the plate surface.

23. The method of claim 21, wherein estimating the volumetric measurement comprises fitting an ellipsoidal surface model to depth frame points associated with the food item by minimizing, via gradient descent, a sum of squared Euclidean distances from food surface vertices to the ellipsoidal model, and computing the volumetric measurement from fitted ellipsoidal parameters.

24. The method of claim 21, wherein estimating the volumetric measurement comprises constructing a triangular mesh from depth frame points associated with the food item and computing the volumetric measurement by summing cross-products of triangle edge vectors integrated over the triangular mesh surface in the gravity-aligned coordinate system.

25. The method of claim 21, further comprising:
performing image segmentation on the depth frame image to identify a plurality of depth-based segments;
selecting a segment having a highest elevation in the gravity-aligned coordinate system as corresponding to a first food item;
applying the trained machine learning classifier to identify the first food item;
removing depth frame points corresponding to the first food item; and
iteratively repeating segment selection, food item identification, and removal for successively lower-elevation segments until the planar reference surface is reached,
wherein each identified segment is classified as a distinct food item contributing to the caloric and nutritional estimate.

26. The method of claim 21, wherein identifying the food item comprises mapping the color image to a feature vector in a shared embedding space using a trained image encoder, computing a cosine similarity between the feature vector and word vectors of candidate food labels in the shared embedding space, and selecting a food label having the highest cosine similarity as the identified food item.

27. The method of claim 21, further comprising:
acquiring, after at least partial consumption of the food item, a post-consumption depth frame image of food remaining on the plate or surface;
applying the gravity-aligned coordinate system to the post-consumption depth frame image;
estimating a post-consumption volumetric measurement of the remaining food by computing, in the gravity-aligned coordinate system, a volume of remaining food surface points above the identified planar reference surface;
computing a consumed volume as a difference between the volumetric measurement estimated prior to consumption and the post-consumption volumetric measurement; and
adjusting the caloric and nutritional estimate based on the consumed volume to determine an actual caloric intake reflecting only consumed food.

28. The method of claim 27, wherein estimating the post-consumption volumetric measurement employs the same plane-fitting algorithm used to identify the planar reference surface prior to consumption, such that both volumetric measurements are computed relative to a common planar reference surface in the gravity-aligned coordinate system.

29. The method of claim 21, further comprising:
receiving GPS location data from the mobile computing device;
identifying a food-serving establishment based on the GPS location data; and
supplementing the nutritional database query with establishment-specific nutritional data retrieved from a restaurant nutritional database associated with the identified establishment,
wherein the caloric and nutritional estimate is determined using at least one of the establishment-specific nutritional data or the estimated volumetric measurement.

30. The method of claim 29, further comprising:
acquiring, by the depth-frame camera, an image of a menu of the identified establishment;
applying Optical Character Recognition (OCR) to the menu image to extract menu item descriptions and any associated caloric information; and
providing the extracted menu item descriptions as contextual constraints to the trained machine learning classifier during food item identification, such that the classifier assigns higher probability to food items corresponding to extracted menu item descriptions.

31. The method of claim 21, further comprising:
acquiring an image of a nutrition label of a food package;
extracting per-serving nutritional information from the nutrition label image using Optical Character Recognition (OCR);
estimating a volume of food to be consumed from the food package using the depth frame image and the estimated volumetric measurement; and
computing the caloric and nutritional estimate by scaling the per-serving nutritional information by a ratio of the estimated consumed volume to a per-serving volume indicated on the nutrition label.

32. The method of claim 21, further comprising storing, in a user-specific data store associated with the mobile computing device, the caloric and nutritional estimate in association with a timestamp, and aggregating stored estimates across a plurality of meals to track cumulative caloric and nutritional intake over a user-defined time period.

33. The method of claim 32, further comprising estimating a ketosis status of the user by determining whether aggregated nutritional intake over the user-defined time period satisfies a ketogenic macronutrient threshold defined by a ratio of carbohydrate intake to a sum of protein intake and fat intake.

34. The method of claim 32, further comprising receiving caloric expenditure data from a fitness tracking application executing on the mobile computing device or a connected device, and computing a net caloric balance by subtracting the caloric expenditure data from the aggregated caloric intake over the user-defined time period.

35. A system for estimating a nutritional and caloric content of food, comprising:
a mobile computing device comprising:
a depth-frame camera configured to capture a color image and a depth frame image of a food item, the depth frame image comprising depth values representing distances between the depth-frame camera and surface points of the food item;
an inertial measurement unit comprising a gravity sensor configured to provide gravity vector data and a gyroscope configured to provide angular rate data;
a non-transitory computer-readable medium storing processor-executable instructions; and
a processor configured to execute the instructions to:
compute a rotation matrix from the gravity vector data and apply the rotation matrix to depth frame coordinates to establish a gravity-aligned coordinate system;
identify a planar reference surface in the gravity-aligned coordinate system by applying a plane-fitting algorithm to depth frame points corresponding to a table surface or a plate surface supporting the food item;
estimate a three-dimensional volumetric measurement of the food item by computing, in the gravity-aligned coordinate system, a volume of food surface points above the identified planar reference surface;
identify the food item by applying a trained machine learning classifier to at least the color image;
determine a caloric and nutritional estimate by querying a nutritional database using the identified food item and the estimated volumetric measurement; and
output the caloric and nutritional estimate to a user interface of the mobile computing device.

36. The system of claim 35, wherein the plane-fitting algorithm comprises a Singular Value Decomposition (SVD) algorithm applied to a matrix of depth frame point coordinates of the table surface or plate surface.

37. The system of claim 35, wherein the processor is further configured to:
acquire, after at least partial consumption of the food item, a post-consumption depth frame image of remaining food;
apply the gravity-aligned coordinate system to the post-consumption depth frame image;
estimate a post-consumption volumetric measurement of the remaining food in the gravity-aligned coordinate system;
compute a consumed volume as a difference between the volumetric measurement prior to consumption and the post-consumption volumetric measurement; and
adjust the caloric and nutritional estimate based on the consumed volume.

38. The system of claim 35, wherein:
the mobile computing device further comprises a GPS receiver; and
the processor is further configured to identify a food-serving establishment from GPS location data received from the GPS receiver, acquire an image of a menu of the identified establishment, apply Optical Character Recognition (OCR) to extract menu item descriptions from the menu image, provide the extracted menu item descriptions as contextual constraints to the trained machine learning classifier, and supplement the nutritional database query with establishment-specific nutritional data.

39. A non-transitory computer-readable medium storing instructions that, when executed by a processor of a mobile computing device, cause the processor to perform operations comprising:
receiving, from a depth-frame camera of the mobile computing device, a color image and a depth frame image of a food item, the depth frame image comprising depth values representing distances from the depth-frame camera to surface points of the food item;
receiving, from an inertial measurement unit of the mobile computing device, orientation data comprising gravity vector data from a gravity sensor and angular rate data from a gyroscope;
computing a gravity-aligned coordinate transformation by deriving a rotation matrix from the gravity vector data;
applying the gravity-aligned coordinate transformation to depth frame coordinates to reorient the depth frame such that the gravity vector is aligned with a vertical axis of the coordinate frame;
identifying a planar reference surface in the reoriented depth frame by applying a plane-fitting algorithm to depth frame points associated with a table or plate supporting the food item;
estimating a volumetric measurement of the food item by computing, in the reoriented depth frame, a volume of food surface points above the identified planar reference surface;
classifying the food item by applying a trained convolutional neural network to the color image;
querying a nutritional database using the classified food item and the estimated volumetric measurement to obtain a caloric and nutritional estimate; and
presenting the caloric and nutritional estimate on a display of the mobile computing device.

40. The non-transitory computer-readable medium of claim 39, wherein the operations further comprise:
receiving, after at least partial consumption of the food item, a second depth frame image of food remaining on the plate or table;
applying the gravity-aligned coordinate transformation to the second depth frame image;
estimating a second volumetric measurement of the remaining food by computing a volume of remaining food surface points above the identified planar reference surface in the reoriented coordinate frame;
computing a consumed volume as a difference between the volumetric measurement and the second volumetric measurement; and
computing an adjusted caloric intake by applying the caloric and nutritional estimate to a ratio of the consumed volume to the volumetric measurement.

41. The non-transitory computer-readable medium of claim 39, wherein the operations further comprise:
receiving GPS location data from the mobile computing device;
identifying a food-serving establishment based on the GPS location data;
receiving an image of a menu of the identified establishment captured by a camera of the mobile computing device;
applying Optical Character Recognition (OCR) to the menu image to extract menu item descriptions and any associated caloric information;
providing the extracted menu item descriptions as contextual constraints to bias the trained convolutional neural network toward food items present on the menu during classification; and
supplementing the nutritional database query with caloric information extracted from the menu image and with establishment-specific nutritional data.
